(19) United States
(12) Patent Application Publication
(10) Pub. No.: US 2015/0290612 A1
(43) Pub. Date: Oct. 15, 2015

(54) ANALYTE SENSOR CHIPS

(71) Applicant: Indian Council of Agricultural Research, New Delhi (IN)

(72) Inventors: Sunil Bhand, Goa (IN); Sudhir Chandra, New Delhi (IN); Hardik Pandya, Delhi (IN); Ruchi Tiwari, New Delhi (IN); Rupesh Kumar Mishra, Goa (IN)

(73) Assignee: Indian Council of Agricultural Research, New Delhi (IN)

(21) Appl. No.: 14/404,362

(22) PCT Filed: Mar. 28, 2013
(86) PCT No.: PCT/IN2013/000205

§ 371 Date: Nov. 26, 2014

(30) Foreign Application Priority Data
Mar. 29, 2012 (IN) 933/MUM/2012

(51) Int. Cl. B01J 19/00; C12Q 1/46; C12Q 1/28; C12Q 1/26; C23C 14/16; C23C 14/34

(52) U.S. Cl. CPC B01J 19/0046; C23C 14/165; C23C 14/34; C12Q 1/28; C12Q 1/26; C12Q 1/46; B01J 2219/00596; B01J 2219/00725

(57) ABSTRACT

The sensor chips, processes and devices enable ultra-sensitive detection/determination, evaluation and quantitative measurement of analytes and are useful for high throughput and miniaturized assays, which enable a user to perform multiple, accurate, experiments in parallel with minimum amount of reagents resulting in low waste generation. The method enables screening of fluid samples to meet regulatory standards. The device comprises a pitted chip having a silicon-based substrate, optionally provided with an integrated heating element, a biosensor and a receptor immobilized on a cross linking element fixed to an inert metal layer in the chip. The analyte is detected up to 5 parts per trillion of the fluid sample and quantitatively measured up to 10 parts per trillion of the fluid sample by the device of the present disclosure.

### FIELD OF THE DISCLOSURE

The present disclosure relates to sensor chips, devices and processes for the manufacture thereof.

### DEFINITIONS

The expression ‘fluid sample’ used in the specification refers to but is not limited to water, waste water, milk, milk products including milk powder, yoghurt, cheese, cream, condensed milk and biological samples including urine, sputum, blood, serum.

The expression ‘pits’ used in the specification refers to but is not limited to wells, cavities, recesses and the like of any shape including square, circular, triangular and the like.

The expression ‘functionalization’ used in the specification refers to but is not limited to the addition of functional groups onto the surface of a material by chemical synthesis methods.

As used, the expression ‘analytes’ in the context of the present disclosure refers to a substance present in a fluid sample which is detected/determined, evaluated and/or quantitatively measured by chemical analysis. The expression “analytes” as used refers to constituents, contaminants, chemical, biochemical and biological species.

The expression ‘chip’ used in the specification refers to but is not limited to a platform for detection/determination, evaluation and/or quantitative measurement of analytes.

The expression ‘biosensor’ used in the specification refers to but is not limited to a device for the detection of analytes and combines a biological component with a physicochemical detector component.

The expression ‘receptor’ used in the specification refers to but is not limited to at least one of native enzymes, stabilized enzymes, genetically modified enzymes, Molecularly Imprinted Polymers (MIPs), antibodies, antigens, aptamers, cells, spores and genetic material.

These definitions are in addition to those expressed in the art.

### BACKGROUND

Pesticides are used in agriculture in order to increase yield and control fungi, insects and weeds. Since the banning of organochlorine, organophosphates and carbamates are the widely used insecticides due to their high activity and relatively low persistence. There have been instances of contamination of ground water, surface water and even bottled water because of the extensive use of pesticides (CSE report 2003). Organophosphate residue levels as high as 75.7 μg/l in bottled drinking water, 26.8 μg/l in soft drinks and 227.8 μg/l in human blood are reported in CSE reports 2002, 2003 and 2005. These reported levels of organophosphates exceed the permissible levels. Among the extensively used pesticides, organophosphate contributes a major share. However, concerns are being raised because of their persistence, bioaccumulation and potential for toxicity in animals and humans. The disadvantage of the use of these insecticides is the contamination of drinking water and foods such as milk and other products.

Due to their acute toxicity and risk to animal and human health, some directives are established to limit the presence of pesticides in water and food resources. Concerning the quality of water for human consumption, the European Council Directive 98/83/EC has set a maximum admissible concentration of 0.1 μg L⁻¹ per pesticide and 0.5 μg L⁻¹ for the total amount of pesticides. CODEX ALIMENTARIUS has set different maximum residual limits (MRL) for food items. Food Safety and Standards Authority of India (FSSAI) has set the limits for pesticides in different matrices. For milk and milk products the tolerance level is 0.01 mg/kg (ppm) for chlorpyrifos and 0.001 mg/kg (ppm) for Malathion (Malathion to be determined and expressed as combined residues of Malathion and malaoxon) in carbonated water.

Milk is one of the widely consumed commodities. Milk and milk products are sometimes contaminated by pesticides due to improper handling and feeding of animals. The major method of entry of pesticide compounds into the body of the milk giving animal is via contaminated feed and fodder. A commonly found pesticide group, organophosphates, is widely used to protect crops in the agricultural sector, and parasite control in domestic animals for veterinary practices. Animals absorb the pesticides as a result of ingestion from residues in their feed and water or by inhalation and dermal absorption during direct/indirect exposure in the course of pest control and thus secrete contaminated milk. These organophosphates are neurotoxins.

The conventional method of Liquid Chromatography with Tandem Mass Spectrometry (LC-MS/MS) is used to detect pesticide residues in milk and milk products. However a disadvantage of the LC-MS/MS method is that the method requires a considerable time period to detect the pesticide residues. Another disadvantage is that a large volume of milk sample is required for the analysis and hence only a few samples of milk can be tested at a time using the LC-MS/MS method. Another disadvantage of the LC-MS/MS method is that the instruments and apparatus for the LC-MS/MS method are very large in size and hence the samples of milk and milk products have to be transported to the location of the laboratory performing the LC-MS/MS method. Also a large infrastructure and skilled manpower is required to operate the instruments and apparatus for the LC-MS/MS method. As a result this conventional method of detection of pesticide residues and impurities in milk and milk products is expensive and time consuming.

Milk contaminated with traces of pesticides is a matter of serious concern. The resulting public concern has created a demand for the development of a reliable, sensitive, simple, and a low cost method for determination of pesticide residues and toxic substances in both milk and milk products. The toxicity of organophosphates justifies the need for accurate and reliable methods to monitor pesticide levels. There is an urgent need for ultra sensitive techniques to rapidly screen these neurotoxic pesticide residues in both milk and milk products.

### Existing Knowledge

There are plenty of works reported in the literature for monitoring or detecting pesticide residues in samples.

United States Patent Application No. 2005054025 discloses a detector where Acetylcholinesterase enzyme is immobilized in a sol-gel or a membrane using a stabilizing solution. The disclosed device is designed for detecting organophosphorous or carbamate compounds. However, US2005054025 is silent on use of pits.

Further, United States Patent Application 20100062455 discloses a kit for rapid detection of cholinesterase inhibitors, for example, snake venoms, organophosphate pesticides, and the nerve gases Sarin, Soman, Tabun, Cyclosarin VR and VX in biological samples such as a blood, plasma, urine or other body fluid of an individual that is exposed to a cholinesterase inhibitor. The disclosed kit employs Acetylcholinesterase enzyme (AChE) immobilized on a surface. The surface is either glass or polymer. In the process as disclosed in aforementioned U.S. patent application, the kit comprises an agent that recovers the Acetylcholinesterase inhibitor from the sample by disrupting the interaction between the Acetylcholinesterase enzyme and Acetylcholinesterase inhibitor. The agent and the Cholinesterase enzyme are then separated from the sample. The activated Acetylcholine inhibitor is then contacted with the Acetylcholinesterase enzyme immobilized on a support. The measurement of the cholinesterase activity to determine the inhibition of Cholinesterase activity is done by using a chromogenic assay mixture that comprises a reagent, for example, Acetylcholine and a Chromogen such as 5,5′-ditho-bis-2-nitrobenzic acid, 4,4-dithiodipyridine or pyridine disulphide or 2,6-Dichloroindophenol.

U.S. Pat. No. 7,217,520 discloses multiwall/microwell assay plates which utilizes a hydrogel polymer, for example, Polyisocynate-functional hydrogel polymer, for immobilization of biological materials such as DNA, RNA, protein and living cells.

Another United States Patent Application 20050287621 discloses surface modification technology for detection of organophosphates such as Sarin and VX. The disclosed chip comprises a substrate coated with a film that includes molecules with a specific ability to combine with an Acetylcholinesterase inhibitor. The substrate as used in the process is a gold coated substrate. The examples of substrates material include glass, quartz, silicon, plastic metal, wafer or polymers. The surface of the substrate is modified with dextran to obtain a hydrophilic surface. Subsequently, molecules with specific ability to combine with the Acetylcholinesterase inhibitors are immobilized on the modified surface. The molecule with a specific binding ability present on the surface of the chip identifies the Acetylcholinesterase inhibitors contained in the sample and combine with them. With the help of a detection element, the surface variation of the chips i.e. the concentration of the adsorbed Acetylcholinesterase inhibitors is quantified.

Another U.S. Pat. No. 5,846,753 discloses sensitive in-situ detection of organophosphates by using chemiluminescence based techniques. A reference article “A fluorescence based biochemical sensing for the detection of organophosphate pesticides and chemical warfare agents” by Viveros et al., Sensors and Actuators B, 2006, Vol 115, pp 150-157, describes a fluorescence based biochemical sensing for the detection of organophosphate pesticides developed in micro molar range.

Further to above disclosed prior-arts, several Lab-on-a-chip devices are also disclosed for ultrasensitive detection of analytes. Lab-on-a-chip for ultrasensitive detection of carbofuran by enzymatic inhibition has been reported in a paper by Llopis X et al., 2009. In this paper an ultrasensitive method to determine toxicity due to pesticides in a glass lab-on-a-chip by means of enzymatic inhibition of Acetylcholinesterase immobilized on magnetic beads is described. For detection of organophosphates, a microchip based method is reported by Wang et al., in reference article “Microchip enzymatic assay of organophosphate nerve agents”, Analytica Chimica Acta, 2004, Vol. 505, pp 183-187. In this method a plastic chip for paraoxon determination is disclosed. Reference article “Disposable biosensor test for organophosphate and carbamate insecticides in milk” by Zhang Y et al., Journal of Agri and Food Chem, 2005, Vol 53, pp 5110-5115, describes detection of organophosphates in milk at ppb level by using electrochemical methods. A high-throughput enzyme assay for organophosphate residues in milk has been presented in a paper by Mishra R K et al., 2010, wherein a 1536 microwell plate has been employed for milk screening.

Although prior art discloses methods for the detection of pesticide residues in a variety of samples, these methods known to allied with number of disadvantages such as poor stability of the immobilized bioactive species on the surface of the chips, lack of portability of the device and instruments and low accuracy in analyte detection in the sample.

Hence there is a need for a device and process that recognizes and quantifies analytes present in fluid samples with maximum accuracy and provides rapid screening of the large number of fluid samples in comparatively shorter time. There is also a need for a device that is economical, portable and does not require highly skilled technicians to operate the same and determine pesticide residues in milk, milk products or fluid samples in general.

### Objects

Some of the objects of the present disclosure aimed to ameliorate one or more problems of the prior art or to at least provide a useful alternative are listed herein below.

An object of the present disclosure is to provide an ultra-sensitive device for detection of analytes present in small quantities of fluid samples.

Another object of the present disclosure is to provide a process and device capable of rapidly screening and quantifying analytes present in fluid samples.

Yet another object of the present disclosure is to provide an economical process and device capable of rapidly screening and quantifying analytes present in fluid samples with maximum accuracy.

Still another object of the present disclosure is to provide a portable device for detection of analytes present in fluid samples.

One more object of the present disclosure is to provide a simple process and device for detection of analytes present in fluid samples that does not require highly skilled technicians.

Still one more object of the present disclosure is to provide a re-usable device for detection of analytes present in fluid samples.

A further object of the present disclosure is to provide sensor chips and methods for the preparation thereof.

Still further object of the present disclosure is to provide a device for evaluating the activity of enzymes and a process for the preparation thereof.

Another object of the present disclosure is to provide a method for surface modification of sensor chips by desired receptors.

Other objects and advantages of the present disclosure will be more apparent from the following description when read in conjunction with the accompanying figures, which are not intended to limit the scope of the present disclosure.

### SUMMARY

In accordance with an aspect of the present disclosure there is provided a device for sensing an analyte in a fluid sample, the device comprising:
(i) a sensor chip comprising:
   a pre-determined array of pits defined on a silicon-based base having a pre-determined thickness; and
   a metal layer of thickness 200-300 nm provided on at least one operative surface of the pits, the metal for the metal layer being at least one metal selected from the group of metals consisting of gold, silver, titanium, rhodium, palladium, platinum and aluminium; and
(ii) a biosensor comprising a cross-linking element fixed to the metal layer, and a set of receptors immobilized on the cross-linking element.

In accordance with one embodiment of the present disclosure, the base comprises a silicon wafer substrate of thickness ranging between 250 and 300 μm, the substrate having at least one oxidized operative surface, and the pits adapted to penetrate the oxidized operative surface and substrate, inner operative surfaces of the pits being oxidized, the metal layer being provided on at least one oxidized operative surface of the pits.

In accordance with another embodiment of the present disclosure, the base comprises:
a substrate selected from the group consisting of a silicon wafer of thickness ranging between 250 and 300 μm, where at least the operative surface is oxidized and glass;
a nickel layer having a thickness ranging between 300 and 400 nm provided on the substrate to at least partially cover the operative surface of the substrate, the nickel layer being in the form of discrete heating elements with terminals to which leads can be attached for externally powering the heating elements;
an electrical insulating layer deposited on the nickel layer; and
a pit defining layer having thickness ranging between 0.2 and 30 μm provided on the electrical insulating layer;
   the metal layer being provided on at least one operative surface of the pits.

Optionally, the device as described herein above comprises a chromium layer having thickness ranging between 50-100 nm is disposed between the metal layer and the operative surfaces of the pits.

In accordance with the present disclosure, the pits typically have varying depth ranging between 0.2 and 30 μm and diameter ranging between 1 mm and 2 mm.

Typically, in accordance with the present disclosure, the cross-linking element is a compound of formula R—X—R′ where R is selected from the group consisting of thiols (—SH), primary amines (NH₂), silica (SiO₂) and phosphate (PO₄⁻³), X is at least one of a repeating unit having 3 to 18 carbon atoms and R′ is selected from the group consisting of cyanides, thiols, amines and carboxyl.

Typically, the cross-linking element is at least one selected from the group consisting of L-Cysteine Hydrochloride, Cysteamine Hydrochloride, 3,3′-Dithiodipropionic acid, 3-Mercaptopropionic acid, 6-Mercaptohexanoic acid, 11-Mercapto-1-undecanol, 12-Mercaptododecanoic acid, 15-Mercaptopentadecanic acid, 16-Mercaptohexadecanoic acid, 3-Aminopropyltriethoxysilane, 16-Phosphonohexadecanoic acid, 11-Phosphonoundecanoic acid and 11-Mercaptoundecanoic acid.

Typically, in accordance with the present disclosure, the receptors are at least one of enzymes, antibodies, antigens, Molecularly Imprinted Polymers (MIPs), aptamers, cells, spores and genetic material.

Typically, the receptors are enzymes selected from the group consisting of native enzymes, stabilized enzymes and genetically modified enzymes. In accordance with an embodiment, the receptors are selected from the group consisting of stabilized choline oxidase, horseradish peroxidase, stabilized Acetylcholinesterase and stabilized Butyrylcholinesterase.

In accordance with another aspect of the present disclosure there is provided a process for making a device for sensing an analyte in a fluid sample, the process comprising the steps of:
providing a silicon-based base having a pre-determined thickness;
forming a pre-determined array of pits on the base; and
depositing a metal layer of thickness 200-300 nm on at least one operative surface of the pits, by at least one of a Direct Current (DC) sputtering and Radio Frequency (RF) sputtering, the metal for the metal layer being at least one metal selected from the group of metals consisting of gold, silver, titanium, rhodium, palladium, platinum and aluminium;
functionalizing the metal layer by using a cross-linking element;
incubating the pits carrying the cross-linking element for a pre-determined period to enable molecular self-assembly of the cross-linking element on the metal layer;
washing the pits with the self-assembled cross-linking element on the metal layer;
activation of the cross-linking element to receive a set of receptors by using a mixture comprising equimolar proportions of 1-ethyl-3-(3-dimethylaminopropyl) carbodiimide and N-hydroxysuccinimide; and
immobilizing the receptors on the activated cross-linking element.

In accordance with an embodiment of the present disclosure, the step of forming a pre-determined array of pits further comprises the steps of:
oxidizing at least the operative surface layer of the base comprising a silicon wafer substrate of thickness ranging between 250 and 300 μm;
etching the oxidized operative surface layer and the substrate; and
oxidizing the inner operative surfaces of the pits;
depositing the metal layer on at least one oxidized operative surface of the pits.

In accordance with another embodiment of the present disclosure, the step of forming a pre-determined array of pits further comprises the steps of:
depositing a nickel layer having a thickness ranging between 300 and 400 nm on the substrate by at least one of Direct Current (DC) and Radio Frequency (RF) sputtering, to at least partially cover the operative surface of the base comprising a substrate selected from the group consisting of a silicon wafer of thickness ranging between 250 and 300 μm, where at least the operative surface is oxidized and glass;
etching by photolithography, the nickel layer in a targeted manner using a mask to form heating elements having terminals;
providing an electrical insulating layer over the formed heating elements;
providing leads for externally powering the heating elements;
forming a pit defining layer having thickness ranging between 0.2 and 30 μm on the silicon dioxide layer, by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating; and
etching the pit defining layer to form a pre-determined array of pits; and
depositing the metal layer on at least one operative surface of the pits.

Again, the step of forming a pre-determined array of pits as described herein above typically comprises the step of etching selected from the group consisting of chemical etching, anisotropic etching and photolithographic etching.

In accordance with yet another aspect of the present disclosure, there is provided a process for evaluating enzyme activity comprising the steps of:
(i) making a device comprising the steps of:
   providing a silicon-based base having a pre-determined thickness, the base being at least one of a silicon wafer substrate of thickness ranging between 250 and 300 μm, where at least the operative surface is oxidized and glass substrate;
   depositing a nickel layer having a thickness ranging between 300 and 400 nm on the oxidized operative surface of the silicon-based layer by at least one of Direct Current (DC) and Radio Frequency (RF) sputtering, to at least partially cover the surface;
   etching by photolithography, the nickel layer in a targeted manner using a mask to form heating elements having terminals;
   providing an electrical insulating layer over the formed heating elements;
   providing leads for externally powering the heating elements; and
   forming a pit defining layer having thickness ranging between 0.2 and 30 μm on the silicon dioxide layer, by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating;
   etching the pit defining layer to form a pre-determined array of pits; and
   depositing a metal layer of thickness 200-300 nm on at least one operative surface of the pits, the metal for the layer being at least one metal selected from the group of metals consisting of gold, silver, titanium, rhodium, palladium, platinum and aluminium;
   functionalizing the metal layer by using the cross-linking element;
   incubating the pits carrying the cross-linking element for a pre-determined period to enable molecular self-assembly of the cross-linking element on the metal layer;
   washing the pits with the self-assembled cross-linking element on the metal layer;
   activation of the cross-linking element to receive the enzyme; and
   immobilizing the enzyme under consideration on the activated cross-linking element,
(ii) adding a first reagent adapted to have a specific reaction with the enzyme, the first reagent being adapted to emit photons by a chemical reaction;
(iii) further incubating the pits repeatedly over pre-determined periods of time and conditions so that the first reagent binds with the enzyme to produce a reaction mixture; and
(iv) comparatively studying the photon count emitted from the reaction mixture in the pits with the photon count emitted over the pre-determined periods of time and conditions.

In accordance with still another aspect of the present disclosure, there is provided a method for detecting at least one analyte in fluid samples, the method comprising the steps of:
(i) a) forming a pre-determined array of first pits on a silicon-based base having a pre-determined thickness, the base constituting a pit defining layer of thickness ranging between 0.2 μm to 30 μm provided on an electrical insulating layer deposited on a nickel layer of thickness ranging between 300 nm and 400 nm adapted to at least partially cover the oxidized operative surface of a silicon wafer substrate of thickness ranging between 250 and 300 μm or the operative surface of a glass substrate, the first pits being coated with a metal layer of thickness 200-300 nm provided on top of an optional chromium layer, the metal for the metal layer being at least one metal selected from the group of metals consisting of gold, silver, titanium, rhodium, palladium, platinum and aluminium, and
   providing a first biosensor comprising a first cross-linking element fixed to the metal layer, and a set of first receptors immobilized on the first cross-linking element;
   b) forming a pre-determined array of second pits on a silicon-based base having a pre-determined thickness, the base constituting a silicon wafer of thickness ranging between 250 and 300 μm, where at least the operative surface layer is oxidized, the second pits being coated with a metal layer of thickness 200-300 nm provided on top of an optional chromium layer, the metal for the metal layer being at least one metal selected from the group of metals consisting of gold, silver, titanium, rhodium, palladium, platinum and aluminium, and
   providing a second biosensor comprising a second cross-linking element fixed to the metal layer, and a set of second receptors immobilized on the cross-linking element;
(ii) adding a fluid sample to at least some of the first pits and incubating for a pre-determined time so that the analyte present in the fluid sample binds with at least some of the first receptors;
(iii) adding a first reagent adapted to have a specific reaction with the first receptors, the first reagent being adapted to emit photons by a chemical reaction, the first reagent is at least one selected from the group consisting of Acetylcholine, Butyrylcholine, Choline, and Hydrogen peroxide;
(iv) further incubating the first pits so that the first reagent binds with at least some of the first receptors which remain unbound during method step (ii) to produce a reaction mixture;
(v) transferring at least a portion of the reaction mixture from the first pits to the second pits;
(vi) adding a second reagent to the second pits to emit photons from the reaction mixture; and
(vii) comparing the photon count emitted from the reaction mixture in the second pits with the photon count emitted by a reference sample.

Typically, in accordance with the present disclosure, the first pits and the second pits are either configured on a single sensor chip or on discrete sensor chips in the method for detecting at least one analyte described herein above.

Additionally, the analyte is detected up to 5 parts per trillion of the fluid sample and quantitatively measured up to 10 parts per trillion of the fluid sample.

### BRIEF DESCRIPTION OF THE ACCOMPANYING DRAWINGS

The disclosure will now be described with the help of the non-limiting accompanying drawings, in which:

FIG. 1a illustrates a schematic representation of a silicon wafer substrate;

FIG. 1b illustrates a schematic representation of the silicon wafer substrate of FIG. 1a that has been subjected to thermal oxidation to form SiO₂ layers;

FIG. 1c illustrates a schematic representation of the silicon wafer substrate of FIG. 1b that has been further subjected to chemical etching to form pits in the operative SiO₂ layer;

FIG. 1d illustrates a schematic representation of the silicon wafer substrate of FIG. 1c that has been still further subjected to anisotropic etching to enable penetration of the pit into the silicon wafer substrate;

FIG. 1e illustrates a schematic representation of the silicon wafer substrate of FIG. 1d with an oxidized pit;

FIG. 1f illustrates a schematic representation of the silicon wafer substrate of FIG. 1e with a metal coating on the oxidized pit;

FIG. 2 illustrates a schematic representation of a sensor chip fabricated on an oxidized silicon substrate;

FIG. 3a illustrates an optical photograph of an array of pits on a sensor chip fabricated on an oxidized silicon wafer;

FIG. 3b illustrates an optical photograph of an array of pits on a sensor chip fabricated on an oxidized silicon wafer with a density of 8×8;

FIG. 4 illustrates an optical photograph of a sensor chip having an integrated microheater;

FIG. 5 illustrates a schematic representation of a sensor chip fabricated on a glass substrate;

FIG. 6 illustrates a portion of a sensor chip having a pattern of microheater (Nickel) heating elements using photolithography;

FIG. 7 illustrates a portion of a device comprising an array of sensor chips with pits created by etching ZnO layer;

FIG. 8 illustrates a schematic representation of the sensor chip of FIG. 5 with a cross-linking element and a receptor on a metal layer;

FIG. 9a illustrates a graphical representation of intensity in ADU of spiked AChE in different fat content milk;

FIG. 9b illustrates a graphical representation of enzyme AChE activity at different temperatures using circular well with heater (CWH2-12);

FIG. 10a illustrates a graphical representation of the calibration curve of methyl paraoxon (MPOx) in milk samples;

FIG. 10b illustrates a graphical representation of the calibration curve of ethyl paraoxon (EPOx) in milk samples;

FIG. 11a illustrates a graphical representation of the calibration curve of methyl parathion (MP) in milk samples;

FIG. 11b illustrates a graphical representation of the calibration curve of carbofuran (CF) in milk samples;

FIG. 12 illustrates a graphical representation of effective degradation of methyl paraoxon (MPOx) using paraoxonase 1 (PON1);

FIG. 13a illustrates a graphical representation of the inhibition curve for pest mix-2;

FIG. 13b illustrates a graphical representation of the inhibition curve for pest mix-174;

FIG. 14 illustrates a graphical representation of the cross validation of results obtained by the sensor chip of FIG. 1 and the chromatographical technique known in the art;

FIG. 15 illustrates a graphical representation of the comparison between stable and non-stable enzyme on the microheater;

FIG. 16 illustrates a graphical representation of the stability in the response of a sensor chip of the present disclosure;

FIG. 17 illustrates a calibration curve of Acetylcholine on the sensor chip;

FIG. 18 illustrates inhibition % by the organophosphate mixture detected at nano level by the sensor chip of the present disclosure;

FIG. 19A and FIG. 19B illustrate a schematic representation for the detection of E. coli and a calibration curve for detection of E. coli in a fluid sample respectively; and

FIG. 20 illustrates a calibration curve obtained for choline in milk using the sensor chip of the present disclosure.

### DETAILED DESCRIPTION OF ACCOMPANYING DRAWINGS

The sensor chips, devices, and processes will now be described with reference to the embodiments shown in the accompanying drawings. The embodiments do not limit the scope and ambit of the disclosure. The description relates purely to the exemplary preferred embodiments of the disclosed structure and its suggested applications.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The description herein after, of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Determination of organophosphates including methyl parathion (MP), methyl paraoxon (MPOx), ethyl paraoxon (EPOx), carbofuran (CF) and combinations thereof is possible using the disclosed sensor chips, processes and devices. The analysis is carried out using an optical detection system [chemiluminescence technique using CCD (Charge Coupled Device) camera or PMT (Photo Multiplier Tubes) based detector]. Parallelly, an economical and reliable screening platform has been fabricated and tested with fluid samples proving efficient performance using sensitive detectors.

The present disclosure demonstrates enzyme based pitted sensor chips, devices and processes which can simultaneously analyze many samples at a time with very small volumes of fluid samples. In a preferred embodiment of the disclosure, the sensor chips have a coating of gold and are fabricated with a reaction area having multiple arrays of pits of same or different sizes and structures, to permit close proximity for reagent interaction, surface modification and immobilization of various biomolecules. The disclosed sensor chips, processes and devices are particularly useful for high throughput and miniaturized assays, which enable a user to perform multiple experiments in parallel with minimum amount of reagents resulting in low waste generation. A combination of sensor chips for detection will have a huge impact on environment via reduction in waste generation. The present disclosure enables creation of single platform based screening devices that are no longer bound to centralized laboratories. A major issue with all lab-on-a-chip or pesticide residue detection chips, known in the art, is a problem related to simple and reliable fluid sample preparation for testing purposes. This is often the bottleneck for sensor-based detection principles, when transferring the detection principle into real life application.

The present disclosed processes have multifold applications, particularly in water industries, dairy industries and milk collection centers enabling screening of large number of fluid samples including water, milk and milk products very rapidly. The sensor chips can also help to determine contamination profiles of organophosphates. In the present disclosure, the fabricated sensor chips are capable of providing various sensitivities on a single platform for priority contaminants.

The sensor chips of the present disclosure are fabricated using Micro-Electro-Mechanical Systems (MEMS) technology for determination of analytes in fluid samples. Specifically, for ease of explanation, the present disclosure refers to determination of organophosphate pesticide (OP) residues in different fat content milk and milk products.

In accordance with one embodiment, the sensor chip is fabricated with an integrated heating element on a silicon substrate. In accordance with another embodiment, the sensor chip is fabricated on a glass substrate. The sensor chip fabricated on glass can be used as a disposable chip, while the sensor chip fabricated on silicon substrate is reusable after washing. Typically, the sensor chip coated with gold is reusable up to nine times.

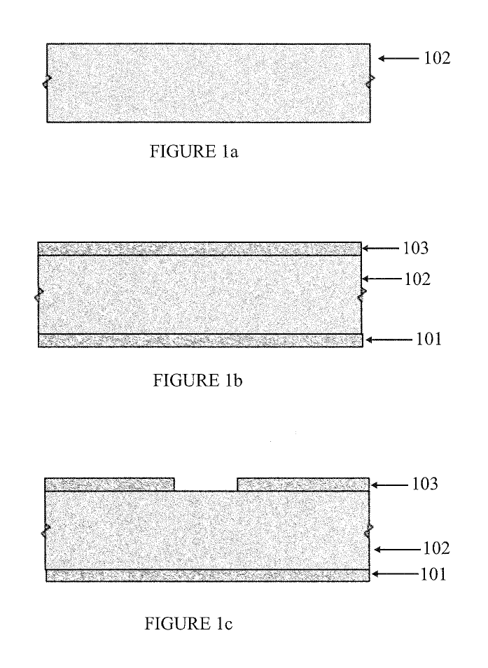

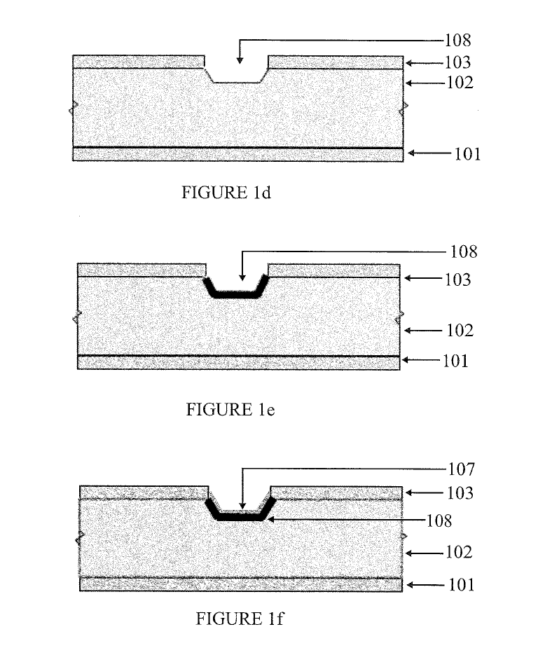

A sensor chip without the integrated heating element, is generally referred hereinafter as Chip B, for ease of explanation. FIG. 1a illustrates a schematic representation of a silicon wafer substrate (102) typically having a thickness of 280 μm. FIG. 1b illustrates a schematic representation of the silicon wafer substrate (102) of FIG. 1a that has been subjected to thermal oxidation to form SiO₂ layers (103). FIG. 1c illustrates a schematic representation of the silicon wafer substrate (102) of FIG. 1b that has been further subjected to chemical etching to form pits in the operative SiO₂ layer. FIG. 1d illustrates a schematic representation of the silicon wafer substrate (102) of FIG. 1c that has been still further subjected to anisotropic etching to enable penetration of the pit (108) into the silicon wafer substrate (102). FIG. 1e illustrates a schematic representation of the silicon wafer substrate (102) of FIG. 1d with an oxidized pit (108). FIGURE if illustrates a schematic representation of the silicon wafer substrate (102) of FIG. 1e with a metal coating (107) on the oxidized pit (108).

In accordance with one aspect of the present disclosure, a device for sensing an analyte in a fluid sample is envisaged. The device comprises:
(i) Chip B, referred herein above, comprises:
   a pre-determined array of pits defined on a silicon-based base having a pre-determined thickness; and
   a metal layer of thickness 200-300 nm provided on at least one operative surface of the pits; and
(ii) a biosensor comprising a cross-linking element fixed to the metal layer, and a set of receptors immobilized on the cross-linking element.

The device comprising Chip B, is generally referred hereinafter as device B, for ease of explanation. The base in device B comprises a silicon wafer substrate (102) of thickness ranging between 250 and 300 μm. The substrate (102) is provided with at least one oxidized operative surface (103) and the pits (108) penetrate the oxidized operative surface (103) and the substrate (102). The operative inner surfaces of the pits (108) are oxidized. A metal layer (107) is provided on at least one oxidized operative surface of the pits (108). The metal for the metal layer is at least one of gold, silver, titanium, rhodium, palladium, platinum and aluminium.

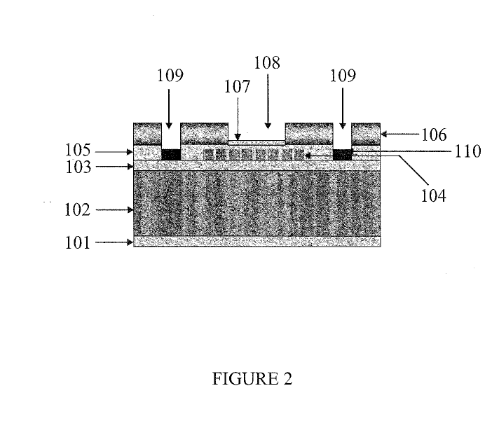

The sensor chip with an integrated heating element as disclosed in FIG. 2 is generally referred as Chip A for ease of explanation. Referring to FIG. 2, a schematic representation of a sensor chip fabricated on a silicon-based substrate having a pre-determined thickness is illustrated. In accordance with one embodiment, the silicon-based substrate of FIG. 2 is a silicon wafer of thickness ranging between 250 and 300 μm. At least the operative surface layer of the silicon wafer substrate is oxidized. The substrate comprises a base substrate layer of silicon dioxide (SiO₂) (101), a middle substrate layer of silicon (Si) (102), and a top substrate layer of SiO₂ (103). A microheater (104) comprising nickel (Ni) as a heating element is then fabricated over the substrate layers. In accordance with one embodiment, the nickel (Ni) layer is in the form of dots. The Ni microheater (104) is insulated by another layer of SiO₂ (105) fabricated over the substrate layers. A pit defining layer (106), typically, zinc oxide (ZnO) or SU-8 is then fabricated over the insulating layer of SiO₂ (105). A pre-determined array of pits (108) is created through the layer of ZnO (106) and the insulating layer of SiO₂ (105). In accordance with one embodiment, the operative inner surfaces of the pits are coated with gold (Au) (107). Alternatively, the metal layer coated on the operative inner surfaces of the pits is silver, titanium, rhodium, palladium, platinum or aluminium. The metal layer is typically of thickness 200-300 nm. Optionally, an adhesive layer, such as chromium layer of thickness 50-100 nm is provided at the bases of the pits for bonding the metal layer. Typically, bottoms of some pits (109) are provided with heater contacts (110) to provide heat to the sensor chips.

A device comprising Chip A, is generally referred hereinafter as device B, for ease of explanation. The device comprises:
(i) Chip A, referred herein above; and
(ii) a biosensor comprising a cross-linking element fixed to the metal layer, and a set of receptors immobilized on the cross-linking element.

In accordance with one embodiment, the base substrate layer of SiO₂ (101) has a thickness of 1 μm, the middle substrate layer of Si (102) has a thickness of 280 μm, and the top substrate layer of SiO₂ (103) has a thickness of 1 μm. The thickness of the microheater (104) is 300 nm. The insulating layer of SiO₂ (105) has a thickness of 1 μm and the layer of ZnO (106) has a thickness of about 0.2 μm to 30 μm. The array of pits (108, 109) measure about 1 mm to 2 mm in diameter and 5 micron to 10 micron in depth.

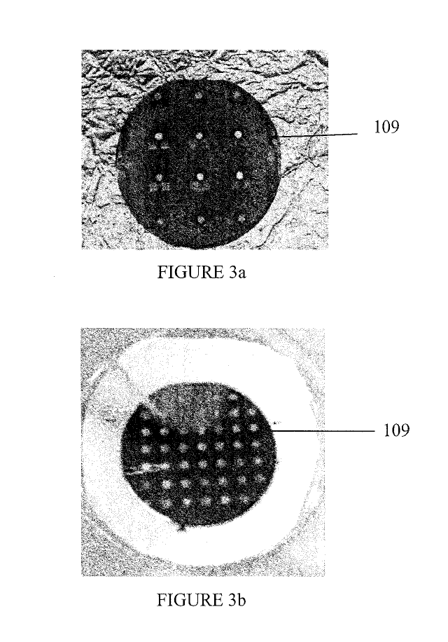

Referring to FIG. 3a, an optical photograph of an array of pits on a sensor chip fabricated on an oxidized silicon wafer is illustrated. Double sided silicon wafers are not required for the fabrication of the sensor chips of the present disclosure, resulting in cost saving. Accordingly, front-to-back mask aligning is also not required for the fabrication of the sensor chips of the present disclosure, resulting in cost saving.

Referring to FIG. 3b, an optical photograph of an array of pits on a sensor chip fabricated on an oxidized silicon wafer with a density of 8×8 is illustrated. Combinations of sixty four pits on the sensor chip arranged in an array of 8×8 are fabricated on the wafer.

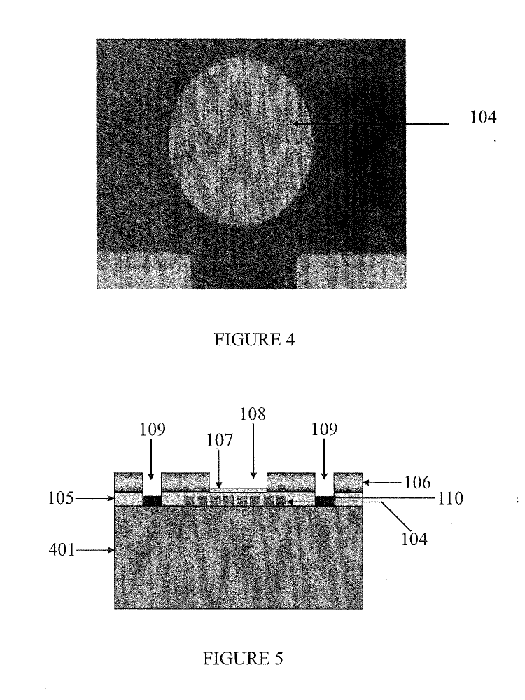

Referring to FIG. 4, an optical photograph of a sensor chip having an integrated microheater is illustrated. In accordance with one embodiment, the microheater comprises nickel (Ni) as heating element (104). In accordance with an embodiment, the thickness of the microheater is 300 nm. The microheater consumes very low power of about 50 mW to 200 mW for achieving 30° C. to 120° C. temperature range. Typically, the sensor chip can efficiently work from 25° C. to 60° C. operating temperature. In accordance with another embodiment, other materials such as nichrome can be used instead of Ni as heating element (104) for microheater fabrication.

Referring to FIG. 5, a schematic representation of a sensor chip fabricated on a glass substrate is illustrated. The substrate comprises a base substrate layer of glass (401). A microheater (104) comprising nickel (Ni) as heating element is then fabricated over the substrate layers. The Ni microheater (104) is insulated by another layer of SiO₂ (105) fabricated over the base substrate layer of glass. A pit defining layer (106), typically zinc oxide (ZnO) or SU-8 is then fabricated over the insulating layer of SiO₂ (105). An array of pits (108) is created through the pit defining layer (106) and the insulating layer of SiO₂ (105). In accordance with one embodiment, the operative inner surface of some pits is coated with gold (Au) (107). Alternatively, the metal layer coated on the operative inner surface of at least some of the pits is silver, titanium, rhodium, palladium, platinum or aluminium. Typically, bottoms of some pits (109) are provided with heater contacts (110). Optionally, an adhesive such as chromium is provided at the bases of the pits for bonding the metal layer. The sensor chip fabricated on glass substrate reduces the cost of sensor fabrication. The sensor chips can be fabricated on glass and the fabrication process can also be upgraded on large sized wafers without changing the dimensions on the masks.

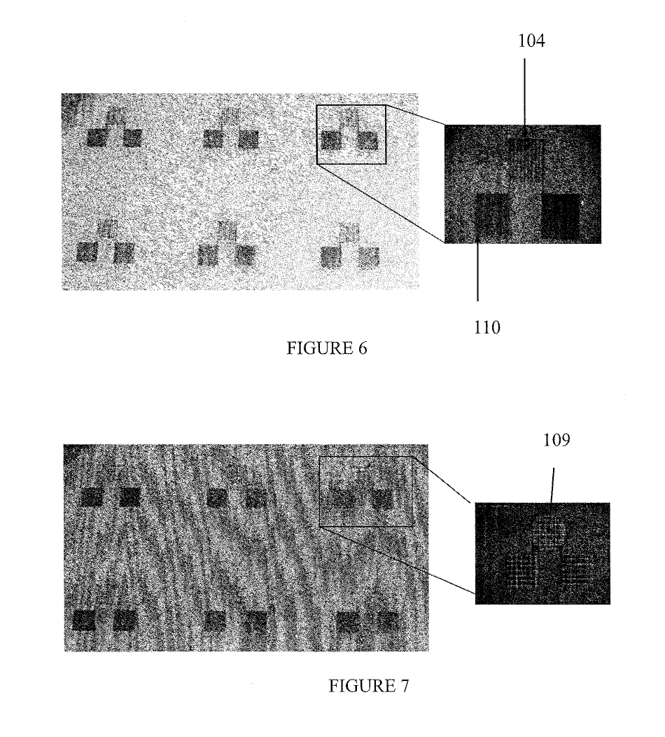

Referring to FIG. 6, a portion of a sensor chip having a pattern of microheater (Nickel) heating elements using photolithography is illustrated. The microheater (104) comprises nickel (Ni) as heating element. The Ni heating element is patterned using photolithography. In accordance with another embodiment, the heating element can be patterned using a metal mask during nickel deposition process. The heating elements of the microheater (104) are insulated by a layer of silicon dioxide (SiO₂) film (105) fabricated over the substrate layers. The SiO₂ film (105) is fabricated by RF sputtering process. RF sputtered SiO₂ is used as an insulating material between the microheater (104) and the layer of ZnO (106). The heater contacts (110) are used to provide heat to the sensor chip. Typically, the heater contact window is opened using photolithography. The layer of ZnO (106) film is fabricated over the insulating layer of SiO₂ (105) by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating, and pits are formed in the zinc oxide layer by chemical etching using 0.5 to 1% dilute hydrochloric acid. In accordance with one embodiment, the thickness of the RF sputtered ZnO film (106) is about 10 microns.

Referring to FIG. 7, a portion of a device comprising an array of sensor chips with pits created by etching ZnO layer is illustrated. The pits (108, 109) are fabricated by chemically etching the pit defining layer (106) of ZnO. Alternatively, anisotropic etching or photolithographic etching is used for etching the pit defining layer to form pits.

In accordance with another aspect of the present disclosure, a process for making a device, for sensing an analyte in a fluid sample comprises the steps of:
providing a silicon-based base having a pre-determined thickness;
forming a pre-determined array of pits on the base; and
depositing a metal layer of thickness 200-300 nm on at least one operative surface of the pits, by at least one of a Direct Current (DC) sputtering and Radio Frequency (RF) sputtering, the metal for the metal layer being at least one metal selected from the group of metals consisting of gold, silver, titanium, rhodium, palladium, platinum and aluminium;
functionalizing the metal layer by using a cross-linking element;
incubating the pits carrying the cross-linking element for a pre-determined period to enable molecular self-assembly of the cross-linking element on the metal layer;
washing the pits with the self-assembled cross-linking element on the metal layer;
activation of the cross-linking element to receive a set of receptors by using a mixture comprising equimolar proportions of 1-ethyl-3-(3-dimethylaminopropyl) carbodiimide and N-hydroxysuccinimide; and
immobilizing the receptors on the activated cross-linking element.

In the process for making Device B comprising Chip B, the step of forming a pre-determined array of pits further comprises the steps of:
oxidizing at least the operative surface layer of the base comprising a silicon wafer substrate of thickness ranging between 250 and 300 μm;
etching the oxidized operative surface layer and the substrate; and
oxidizing the inner operative surfaces of the pits;
depositing the metal layer on at least one oxidized operative surface of the pits.

In the process for making Device A comprising Chip A, the step of forming a pre-determined array of pits further comprises the steps of:
depositing a nickel layer having a thickness ranging between 300 and 400 nm on the substrate by at least one of Direct Current (DC) and Radio Frequency (RF) sputtering, to at least partially cover the operative surface of the base comprising a substrate selected from the group consisting of a silicon wafer of thickness ranging between 250 and 300 μm, where at least the operative surface is oxidized and glass;
etching by photolithography, the nickel layer in a targeted manner using a mask to form heating elements having terminals;
providing an electrical insulating layer over the formed heating elements;
providing leads for externally powering the heating elements;
forming a pit defining layer having thickness ranging between 0.2 and 30 μm on the silicon dioxide layer, by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating; and
etching the pit defining layer to form a pre-determined array of pits; and
depositing the metal layer on at least one operative surface of the pits.

The step of forming a pre-determined array of pits comprises the step of etching selected from the group consisting of chemical etching, anisotropic etching and photolithographic etching.

The step of depositing a metal layer optionally comprises the step of providing a adhesive layer, typically, chromium layer of thickness ranging between 50-100 nm between the metal layer and the operative surfaces of the pits.

The process as described herein above includes the steps of depositing the metal layer by Direct Current (DC) or Radio Frequency (RF) sputtering; targeting the pits through a masking template and bonding the metal layer to the base of the pits using an adhesive.

The step of forming a pit defining layer typically includes the steps of depositing zinc oxide by Direct Current (DC) or Radio Frequency (RF) sputtering to form the zinc oxide layer; and chemical etching using 0.5 to 1% dilute hydrochloric acid to form pits in the zinc oxide layer.

The step of making the biosensor includes the steps of:
functionalizing the metal layer by using the cross-linking element;
incubating the chip carrying the cross-linking element for a pre-determined period to enable molecular self-assembly of the cross-linking element on the metal layer;
washing the chip with the self-assembled cross-linking element on the metal layer in the pits;
activation of the cross-linking element to receive the receptors; and
immobilizing the receptors on the activated cross-linking element.

The step of activation stated herein above is carried out by using a mixture comprising equimolar proportions of 1-ethyl-3-(3-dimethylaminopropyl) carbodiimide and N-hydroxysuccinimide.

The cross-linking element is typically a compound of formula R—X—R′ where R is selected from the group consisting of thiols (—SH), primary amines (NH₂), silica (SiO₂) and phosphate (PO₄³), X is at least one of a repeating unit having 3 to 18 carbon atoms and R′ is selected from the group consisting of cyanides, thiols, amines and carboxyl.

The receptors are at least one of enzymes, antibodies, antigens, Molecularly Imprinted Polymers (MIPs), aptamers, cells, spores and genetic material.

Alternatively, the receptors are enzymes selected from the group consisting of native enzymes, stabilized enzymes and genetically modified enzymes.

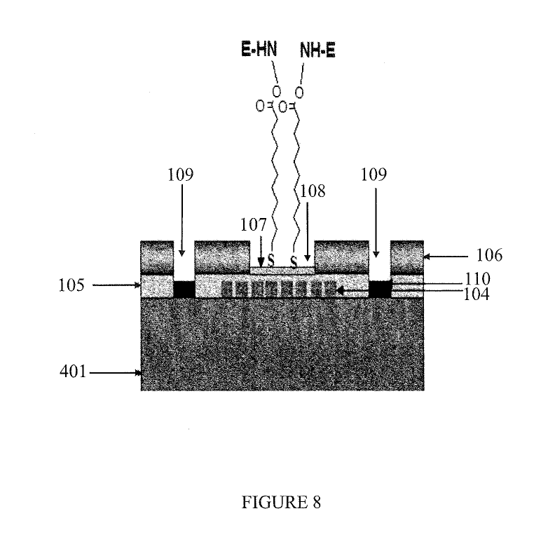

FIG. 8 illustrates a schematic representation of the sensor chip of FIG. 5 with a cross-linking element and a receptor on the metal layer (107), wherein E represents a receptor.

Typically, the cross-linking element is selected from the group consisting of L-Cysteine Hydrochloride, Cysteamine Hydrochloride, 3,3′-Dithiodipropionic acid, 3-Mercaptopropionic acid, 6-Mercaptohexanoic acid, 11-Mercapto-1-undecanol, 12-Mercaptododecanoic acid, 15-Mercaptopentadecanic acid, 16-Mercaptohexadecanoic acid, 3-Aminopropyltriethoxysilane, 16-Phosphonohexadecanoic acid, 11-Phosphonoundecanoic acid and 11-Mercaptoundecanoic acid.

Specifically, experiments were conducted using stabilized choline oxidase and horseradish peroxidase as receptors in Chip B and stabilized Acetylcholinesterase (AChE) or stabilized Butyrylcholinesterase (BuChE) as receptor in Chip A. Likewise, for Chip B, the cross-linking element used was 11-Mercaptoundecanoic acid and for Chip A, the cross-linking element was 11-Mercaptoundecanoic acid when used with gold metal layer or 16-phosphonohexadecanoic acid when used with Aluminium metal layer.

The devices using Chip A and Chip B herein above also find application for evaluating enzyme activity and its thermal stability, biological determination, biological contamination, and the like.

In accordance with still another aspect of the present disclosure, a system and process for detecting analytes in a fluid sample, is disclosed, the system comprising: a device A, a device B, means for transferring at least a portion of a fluid sample from the device A to the device B; and an optical detector co-operating with the device B.

In accordance with yet another aspect of the present disclosure, the sensor chips as disclosed are used for detecting at least one analyte in a fluid sample. The method comprises the steps of:
(i) a) forming a pre-determined array of first pits on a silicon-based base having a pre-determined thickness, the base constituting a pit defining layer of thickness ranging between 0.2 μm to 30 μm provided on an electrical insulating layer deposited on a nickel layer of thickness ranging between 300 nm and 400 nm adapted to at least partially cover the oxidized operative surface of a silicon wafer substrate of thickness ranging between 250 and 300 μm or the operative surface of a glass substrate, the first pits being coated with a metal layer of thickness 200-300 nm provided on top of an optional chromium layer, the metal for the metal layer being at least one metal selected from the group of metals consisting of gold, silver, titanium, rhodium, palladium, platinum and aluminium, and
   providing a first biosensor comprising a first cross-linking element fixed to the metal layer, and a set of first receptors immobilized on the first cross-linking element;
   b) forming a pre-determined array of second pits on a silicon-based base having a pre-determined thickness, the base constituting a silicon wafer of thickness ranging between 250 and 300 μm, where at least the operative surface layer is oxidized, the second pits being coated with a metal layer of thickness 200-300 nm provided on top of an optional chromium layer, the metal for the metal layer being at least one metal selected from the group of metals consisting of gold, silver, titanium, rhodium, palladium, platinum and aluminium, and
   providing a second biosensor comprising a second cross-linking element fixed to the metal layer, and a set of second receptors immobilized on the cross-linking element;
(ii) adding a fluid sample to at least some of the first pits and incubating for a pre-determined time so that the analyte present in the fluid sample binds with at least some of the first receptors;
(iii) adding a first reagent adapted to have a specific reaction with the first receptors, the first reagent being adapted to emit photons by a chemical reaction;
(iv) further incubating the first pits so that the first reagent binds with at least some of the first receptors which remain unbound during method step (ii) to produce a reaction mixture;
(v) transferring at least a portion of the reaction mixture from the first pits to the second pits;
(vi) adding a second reagent to the second pits to emit photons from the reaction mixture; and
(vii) comparing the photon count emitted from the reaction mixture in the second pits with the photon count emitted by a reference sample.

In accordance with a further aspect of the present disclosure, sensor chips as disclosed are used for evaluating activity of enzymes. The process for evaluating activity of an enzyme comprises the steps of:
(i) making a device comprising the steps of:
   providing a silicon-based base having a pre-determined thickness, the base being at least one of a silicon wafer substrate of thickness ranging between 250 and 300 μm, where at least the operative surface is oxidized and glass substrate;
   depositing a nickel layer having a thickness ranging between 300 and 400 nm on the oxidized operative surface of the silicon-based layer by at least one of Direct Current (DC) and Radio Frequency (RF) sputtering, to at least partially cover the surface;
   etching by photolithography, the nickel layer in a targeted manner using a mask to form heating elements having terminals;
   providing an electrical insulating layer over the formed heating elements;
   providing leads for externally powering the heating elements; and
   forming a pit defining layer having thickness ranging between 0.2 and 30 μm on the silicon dioxide layer, by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating;
   etching the pit defining layer to form a pre-determined array of pits; and
   depositing a metal layer of thickness 200-300 nm on at least one operative surface of the pits, the metal for the layer being at least one metal selected from the group of metals consisting of gold, silver, titanium, rhodium, palladium, platinum and aluminium;
   functionalizing the metal layer by using the cross-linking element;
   incubating the pits carrying the cross-linking element for a pre-determined period to enable molecular self-assembly of the cross-linking element on the metal layer;
   washing the pits with the self-assembled cross-linking element on the metal layer;
   activation of the cross-linking element to receive the enzyme; and
   immobilizing the enzyme under consideration on the activated cross-linking element,
(ii) adding a first reagent adapted to have a specific reaction with the enzyme, the first reagent being adapted to emit photons by a chemical reaction;
(iii) further incubating the pits repeatedly over pre-determined periods of time and conditions so that the first reagent binds with the enzyme to produce a reaction mixture; and
(iv) comparatively studying the photon count emitted from the reaction mixture in the pits with the photon count emitted over the pre-determined periods of time and conditions.

Enzyme activity was particularly tested on the following enzymes:
Acetylcholinesterase (AChE) enzyme from Drosophila melanogaster and Electrophorus electricus (electric eel)—native and stabilized;
Butyrylcholinesterase (BuChE) enzyme—native and stabilized;
choline oxidase enzyme (ChOx)—Alcaligenes species and stabilized ChOx;
horseradish peroxidase enzyme (HRP);
glucose oxidase enzyme (GOx); and
Paraoxonase 1.

The biochemical reactions in accordance with an embodiment of the present disclosure are as follows,

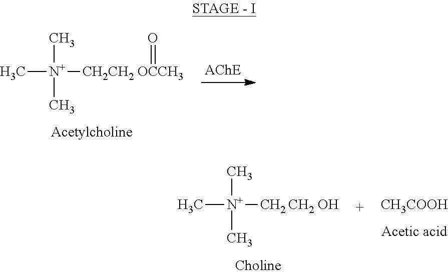

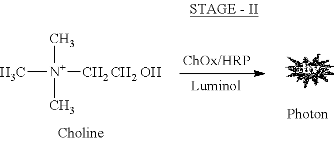

In accordance with another embodiment, a receptor Butyrylcholinesterase (BuChE) enzyme is immobilized on the sensor chip and is used for analysis of pesticide residues.

### Experimental Data

A receptor typically, Acetylcholinesterase (AChE) enzyme is coupled to a sensor chip using an immobilization protocol. The sensor has been tested successfully for MPOx, EPOx, carbofuran (CF) and MP and combinations thereof, in milk and also in water. Reference pesticide mixtures have also been tested in milk. The processes of the present disclosure have been cross validated against the approved conventional method LC-MS/MS and results confirm that the disclosure is ultra-sensitive and determine pesticide residues at nano levels in milk and water.

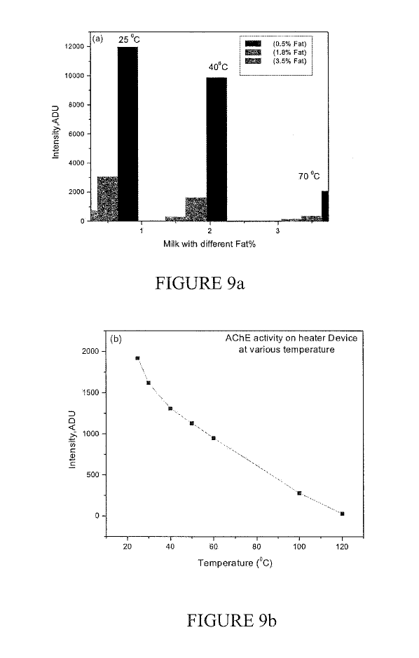

Referring to FIG. 9a, a graphical representation of intensity in ADU of spiked AChE in different fat content milk is illustrated. Three different fat content milk samples comprising 0.5% fat, 1.8% fat, and 3.5% fat, were successfully evaluated for thermal stability of enzyme in milk, on a sensor chip with integrated heating element. First bar shows experiments conducted at 25° C. with different fat milk such as 0.5%, 1.8% and 3.5%. Similarly, second and third bar shows experiments conducted at 40° C. and 70° C. respectively.

Referring to FIG. 9b, a graphical representation of enzyme AChE activity, in milk having 0.5% fat content, at different temperatures using circular well with heater (CWH2-12) is illustrated. The experiment was performed on AChE having a concentration of 0.09 IU, using AChCl of concentration 1 mM, at different temperatures using a circular well with a microheater. The stabilized enzyme was tested and found active up to 40° C. without losing any noticeable activity.

In accordance with another embodiment, the milk samples of varying fat content stored at 4° C. were analyzed for organophosphate residues using the sensor chip A. The milk sample is brought to a working temperature (tunable as per the storage condition and fat content) using the sensor chip A with an integrated microheater. After heating the milk sample on the sensor chip A, the enzyme is added to the sensor chip A and rest of the procedure is followed as per the stages I and II described above.

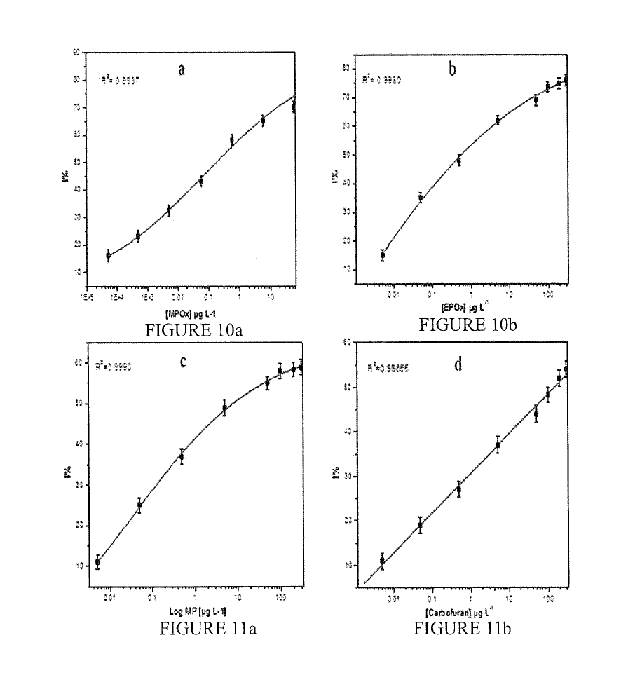

Referring to FIGS. 10a, 10b, 11a and 11b, a graphical representation of the calibration curve of methyl paraoxon (MPOx), the calibration curve of ethyl paraoxon (EPOx), the calibration curve of methyl parathion (MP) and the calibration curve of carbofuran (CF), respectively are illustrated. Milk samples containing 0.5% fat, 1.8% fat and 3.5% fat were analyzed. Milk samples were only filtered and diluted prior to analysis. Matrix matching studies were carried out by preparing different dilutions of milk in Phosphate Buffer (PB). Milk samples were spiked with individual pesticides MPOx, MP, EPOx and CF and their mixture. The sensor chip without heating element was tested for four potent pesticides, MPOx, MP, EPOx and CF and combinations thereof. Enzymatic activity in the sensor chip was measured and compared to an enzymatic activity in a reference control solution to determine the concentration of the pesticide present in the milk samples.

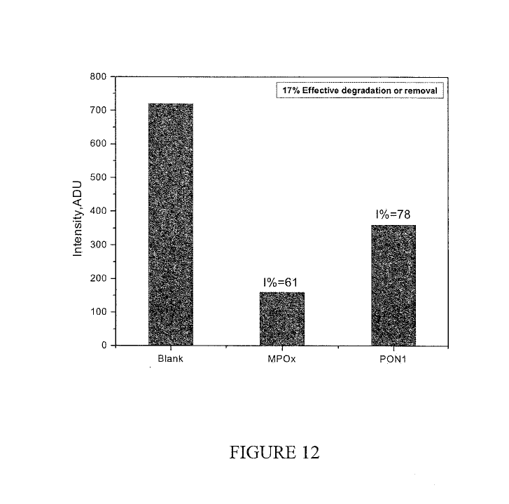

FIG. 12 illustrates effective degradation of methyl paraoxon (MPOx) using paraoxonase 1 (PON1) in milk samples. Enzyme PON1 was immobilized in one of the pits and Acetylcholinesterase enzyme (AChE) was immobilized in at least one of the other pits. A known pesticide solution was incubated in both the pits and the activity was measured using the generated photon count.

In addition to detect pesticide residues, the enzymatic method may be used to quantify pesticide residues. The photon count generated by the optical detector is proportional to the degree to which the pesticides inhibit enzyme with respect to a reference.

These pesticides were tested in milk samples having three different fat contents, within a very short analysis time of 15 minutes. The sensor chip showed an excellent response for concentration level as low as 5 parts per trillion (ppt) in milk. The sensor chip is capable of quantifying the analyte as low as 10 parts per trillion (ppt) in milk.

In accordance with another embodiment, at least a portion of the milk sample comprising a pesticide (1 μL) is added to a reagent solution comprising an enzyme and a reagent where enzyme is of a type inhibited by the pesticide.

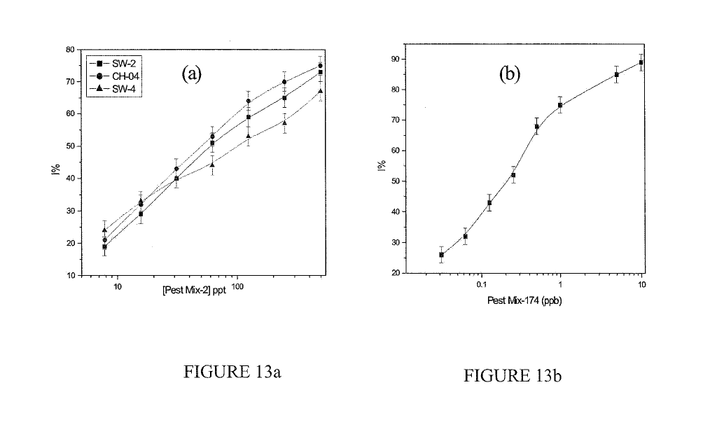

Referring to FIGS. 13a and 13b, a graphical representation of the inhibition curves for pest mix-2 and pest mix-174 is illustrated. Particularly in FIG. 13a, SW-2, CH-04 and SW-4 represent sensor chips having different dimensions. The sensor chip was successfully tested for the reference pesticides, (Pesticide mixture 2 and mixture 174 from Dr. E from Germany) at nano gram level. The pesticide mix 2 contains diazinon, ethion, malathion, parathion ethyl and parathion methyl of concentration 10 ng/μL. The pesticide mix 174 containing azinphos-methyl, bromophos-ethyl, chlorpyriphos-methyl, demeton-s-methyl, diazinon, ethion, fenitrothion, malaoxon, malathion, methamidophos, methidathion, paraoxon methyl, phosphamidon and trichlorphon has been tested on the sensor chip to ascertain the Toxicity Equivalence (TEQ) of pesticide residues and the total inhibition in milk.

In addition the sensor chip was also tested for analysis of organophosphates in presence of other co-contaminants such as Aflatoxin M1 (AFM1), organochlorine pesticide residues such as atrazine, simazine and 2,4-D and no interference was found.

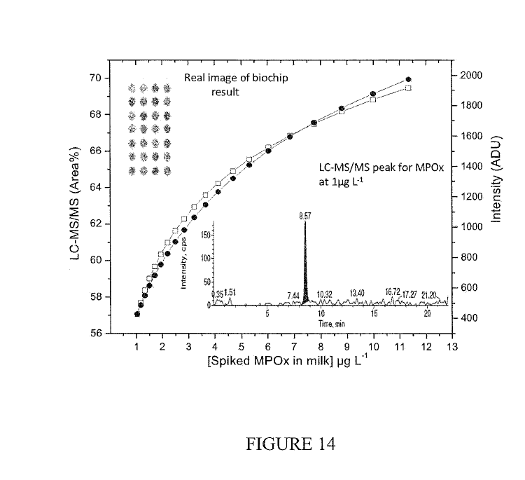

FIG. 14 illustrates a graphical representation of the cross validation of results obtained by the sensor chip of FIG. 1 and the chromatographical technique known in the art. The method for detecting pesticide residues in milk and milk products is successfully cross validated using the LC-MS/MS technique. The recovery of individual organophosphates and mixture of organophosphates using the sensor chip is at par with those found using approved LC-MS-MS method for the same batch of fluid samples. High throughput analysis of pesticide residues in milk and milk products has been accomplished using heating and non-heating sensor chips.

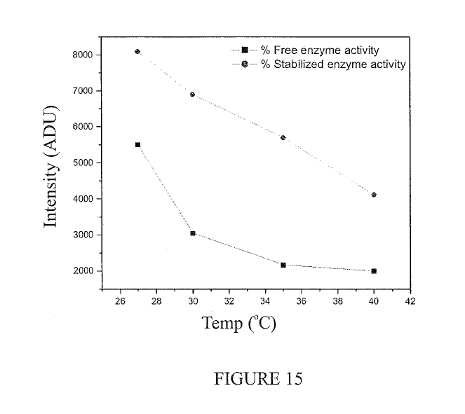

FIG. 15 illustrates a graphical representation of the comparison between stable and non-stable enzyme on the microheater. A fixed quantity of the stabilized AChE was dispensed on the chip surface and was dried at room temperature. Stabilized AChE forms thin film like layer at the bottom of the pits of the chip of the present disclosure. Subsequently, a known volume of inhibitor (in PB or Milk) was added to the pits and incubated for 10 min. The reaction was followed by addition of a second reagent. The number of photons (photon count) emitted was recorded.

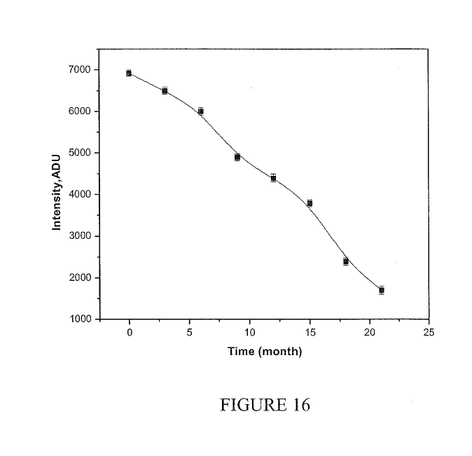

FIG. 16 illustrates a graphical representation of the stability in the response of an sensor chip of the present disclosure. For stability of the sensor chip and to evaluate the immobilization of enzyme on different sensor chips, two different batches of same sensor chips were examined. Inter and intra-batch chip performance was highly reproducible.

### Exemplary Experiment 1 Step 1

(i) Preparation of Reagent:

The Acetylcholine chloride (AChCl) (181 mg solids) from Sigma Aldrich, lot no 075K2606 was dissolved in 10 ml of PB, pH 7.4 and was mixed. This standard solution was further diluted with a buffer to make different AChCl solutions.

The butyrylcholine chloride (209 mg solids) from Sigma Aldrich, lot no 031K1681 was dissolved in 10 ml of phosphate buffer, pH 7.4 and was mixed. This standard solution was further diluted with the buffer to make different BuChCl solutions.

(ii) Pesticide Standards and Certified Standards

Stock Pesticide Preparations:

Stock solutions (1 mg mL-1) of MPOx (Reidel-de Haen, lot: 4062X), EPOx (Fluka, lot no: 7302X) and MP (Reidel-de Haen, lot: 2317X), were prepared in 5% acetonitrile, whereas carbofuran (Reidel-de Haen, lot: 8088X), was prepared using 60% acetonitrile. Aliquots were kept at 2-8° C. Pesticide standards were diluted in Phosphate Buffer (PB) on the day of use. Pesticide mix reference solutions were procured from Dr. E. The concentration of pest mix-2 (lot no: 00709CY) was 10 ng/μL (10 ppm) and Pest Mix-174 (lot no: 00622EA) was 200 ng/μL (200 ppm).

A stock solution of 1 ppm was prepared by adding 10 μL of pest mix-2 in 90 μL of PB. This standard solution was further diluted with buffer to make different working solutions.

The same procedure was followed for pest mix-174. A stock solution of 2 ppm was prepared by adding 10 μL of pest mix-2 in 990 μL of PB. This standard solution was further diluted with buffer to make different working solutions.

(iii) Preparation of Enzyme Solutions:

Butyrylcholinesterase from Equine serum, Sigma Aldrich (lot no: 026K705d): Dissolve 1 mg (7.4 IU) lyophilized powder of BuChE in 1000 μL of PB pH 7.4.

Acetylcholinesterase from electrophorus electricus electric eel Sigma Aldrich (lot no: 047K7010): Dissolve 0.5 mg of lyophilized powder of AChE (259 IU) in 100 μL of PB pH 7.4.

Choline oxidase from Alkaligenes species Sigma Aldrich (lot no: 048K1154) and from Gwent batch no: #2100127: Dissolve 1 mg (10 IU) lyophilized powder of ChOx in 500 μL of PB pH 7.4.

Peroxidase from Horseradish Sigma Aldrich (lot no: 090M77113): Dissolve 1 mg (260 IU) lyophilized powder of HRP in 2000 μL of PB pH 7.4.

Luminol from Sigma Aldrich (lot no: 91H38561): To prepare 1 mM of luminol solution, dissolve 4 mg of powder in 18 ml of PB, pH 7.4 and 2 ml of 0.1M NaOH.

### Step II

(i) Functionalization of Chip

Firstly, 5 mM-10 ml, 11-Mercaptoundecanoic acid solution (for this weigh 10.95 mg in 10 ml Absolute ethanol) was prepared.

4 μl of prepared 11-Mercaptoundecanoic acid solution was added on chip pits and incubated for 24 hours at room temperature.

After the incubation, the chip was thoroughly washed with ethanol three times.

After washing with ethanol, EDC (100 mM)-NHS (100 mM) mixture was added in the ratio of 1:1 (200 μl:200 μl) and again incubated for 3 hours.

After 3 hours of incubation, the chip was washed with 300 μl of PB pH 7.4, 0.1M.

After activation of the pits on the chip, enzymes 2.5 μl of ChOx (0.05 U) and 1 μl of HRP (0.08 U) were co-immobilized on the chip and left for 3 hours.

After 3 hours, the chip was used to construct a calibration curve.

### Step III

Milk Screening Procedure:

The analysis of organophosphate pesticide residues using the device was accomplished within 15 min. The analysis was carried out in three steps: In a first step, the stabilized AChE was coupled to the chip (A), after this step, the inhibitor test solution (ca MPOx) was added to the chip (A) and incubated for 10 min. In a second step, chip (B) comprising co-immobilized bi-enzyme (ChOx and HRP) was used. In the chip A, AChCl was added subsequent to the incubation step described in stage I. After a two-minute reaction, the product was collected from Chip A and transferred to chip B. After a two min reaction on Chip B, the product was dispensed to a 1536 micro-well plate and luminol was added to complete the reaction and measure the intensity using an optical detector. The blank, reference and the fluid sample intensities were compared to determine the inhibition of the activity of AChE and subsequent calibration curves were drawn.

### Exemplary Experiment 2

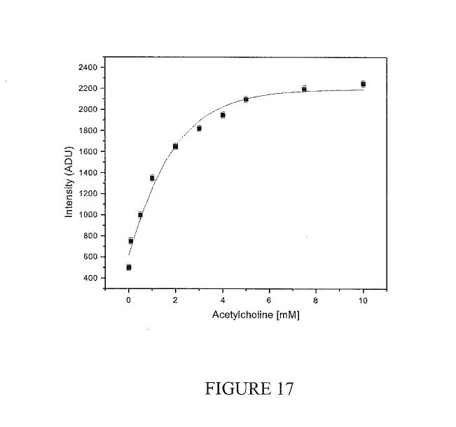

FIG. 17 illustrates a calibration curve of Acetylcholine on the sensor chip. The calibration curve was constructed for Acetylcholine by spiking a known concentration of Acetylcholine chloride (AChCl) in diluted milk (0.5% fat containing milk) ranging from 0.01 to 10 mM (10, 7.5, 5, 4, 3, 2, 1, 0.5, 0.1, 0.01 mM). The reacted choline from this pit was transferred to another pit, having co-immobilized ChOx and HRP, for generation of photons. The obtained photon count was used for the calibration curve.

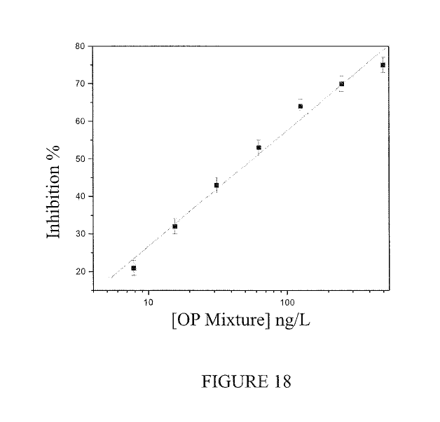

FIG. 18 illustrates inhibition % by the organophosphate mixture detected at nano level by the sensor chip of the present disclosure.

The present disclosed process has been validated against the conventional method.

TABLE 1
Recovery studies in milk using the device of the present disclosure.

| No of assay | Fluid samples | |
|---|---|---|
| performed | Analyzed | Results |
| 10 | Zero Control | All negative (recoveries are |
| | | below 100%) |
| 08 | MPOx at 0.05 ppb | 6 negative and 2 positive |
| 08 | MPOx at 0.5 ppb | 7 negative and 1 positive |
| 08 | MPOx at 5 ppb | 6 negative and 2 positive |

*Positive means = recoveries are more than 100%
*Negative means = recoveries are about 100%
Typically acceptable recovery range of analyte is 80-120%

TABLE 2
Comparison of recoveries obtained for MPOx using LC-
MS/MS and the sensor chip of the present disclosure.

| | Conc. | Conc. found | % | | % |
|---|---|---|---|---|---|
| | Spiked | (μg L⁻¹) | Recovery | Conc. found | Recovery |
| Sr. | (μg L⁻¹) | (LC- | (LC- | (μg L⁻¹) | (sensor |
| No. | (MPOx) | MS/MS) | MS/MS) | sensor chip | chip) |
| 1 | 9.28 | 8.61 | 92.8 | 9.15 | 98.60 |
| 2 | 5.38 | 5.78 | 107.6 | 5.33 | 99.20 |
| 3 | 2.58 | 2.67 | 103.2 | 2.60 | 100.78 |
| 4 | 1.52 | 1.85 | 121.6 | 1.50 | 99.20 |
| 5 | 1.26 | 2.52 | 201.6 | 1.24 | 99.00 |
| 6 | 0.1 | — | ND | 0.097 | 97.00 |

*ND = Not detected

TABLE 3
Recoveries obtained for pest mix-2 in different milk samples
using the sensor chip of the present disclosure.

Developed assay for pest mix-2's analysis

| | [Pest-mix-2] | [Pest-mix-2] | | |
|---|---|---|---|---|
| | spiked | Found | | |
| Matrix | (μg L⁻¹) | (μg L⁻¹) | Recovery % | SD |
| Milk-1 | 0.125 | 0.123 | 99.05 | 3.18 |
| Milk-2 | 0.125 | 0.122 | 98.06 | 3.56 |
| Milk-3 | 0.0625 | 0.059 | 95.03 | 3.02 |
| Milk-4 | 0.0625 | 0.0621 | 99.43 | 3.30 |
| Milk-5 | 0.0312 | 0.0305 | 97.80 | 2.85 |
| Milk-6 | 0.0312 | 0.0307 | 98.60 | 3.02 |

TABLE 4
Recoveries obtained for pest mix-174 in different milk samples
using the sensor chip of the present disclosure.

| | OPs analysis on chip | | |
|---|---|---|---|
| | Pest mix-174- | Pest mix-174 | |
| | spiked | found | |
| Matrix | (μg L⁻¹) | (μg L⁻¹) | % Recovery |
| Milk-1 | 0.5 | 0.485 ± 0.3 | 97.0 |
| Milk-2 | 0.5 | 0.488 ± 0.1 | 97.6 |
| Milk-3 | 0.5 | 0.490 ± 0.4 | 98.0 |
| Milk-4 | 1 | 0.94 ± 0.61 | 94.0 |
| Milk-5 | 1 | 0.95 ± 0.40 | 95.0 |
| Milk-6 | 1 | 0.947 ± 0.30 | 94.7 |

Measurements on Chips:
1) The inhibition measurements were carried out in milk using pest mix-174 at a concentration level of 0.5 ppb (EU cut off). The known concentration (0.5 ppb) of pest mix was spiked in milk samples and incubated with AChE on chip A for 10 minutes. After incubation, 1 mM AChCl was dispensed on pits of chip A. AChCl and AChE reacts and choline was produced during this reaction. The produced choline was collected from chip A using a micropipette and dispensed on the other chip (chip B) where ChOx and HRP were co-immobilized. Choline reacts with the co-immobilized ChOx and HRP The product was transferred to a 1536-micro well plate with subsequent addition of luminol. Addition of luminol generates photons which were recorded by a recorder and the differences in the photon count were tabulated.

TABLE 5
The intensity recorded during the analysis of pest mix-174 in
milk at 0.5 ppb (EU cut off) during four different measurements
performed on a single chip at four different time periods.

| | Photon count | Photon count |
|---|---|---|
| Measurement | (Reference) | (Sample) |
| 1 | 7780 | 4350 |
| 2 | 7590 | 4490 |
| 3 | 7610 | 4390 |
| 4 | 7740 | 4400 |

Measurements on Chips:
2) The inhibition measurements were carried out in milk using pest mix-174 at different concentration level (500, 250, 125, 62.5, 31.35, 15.62 and 7.81 ng/mL). Three known concentrations of pest mix were spiked in milk samples and incubated with AChE on chip A for 10 minutes. After incubation, 1 mm AChCl was dispensed on pits of chip. AChCl and AChE reacts and choline was produced during this reaction. The produced choline was collected from the chip (Chip A) using a micropipette and dispensed on the other chip (Chip B) where ChOx and HRP were co-immobilized. Choline reacted with the co-immobilized ChOx and HRP. The product was transferred to a 1536-micro well plate with subsequent addition of luminol. Addition of luminol generates photons which were recorded by a recorder and the differences in the photon count were tabulated.

TABLE 6
Photon count obtained for AChE inhibition using pest mix-174

| Reference | 500 ng/L | 250 ng/L | 125 ng/L | 62.5 ng/L | 31.35 ng/L | 15.62 ng/L | 7.81 ng/L |
|---|---|---|---|---|---|---|---|
| 5000 | 1420 | 1800 | 2070 | 2530 | 2950 | 3430 | 3930 |

### Exemplary Experiment 3

### Detection of E. coli

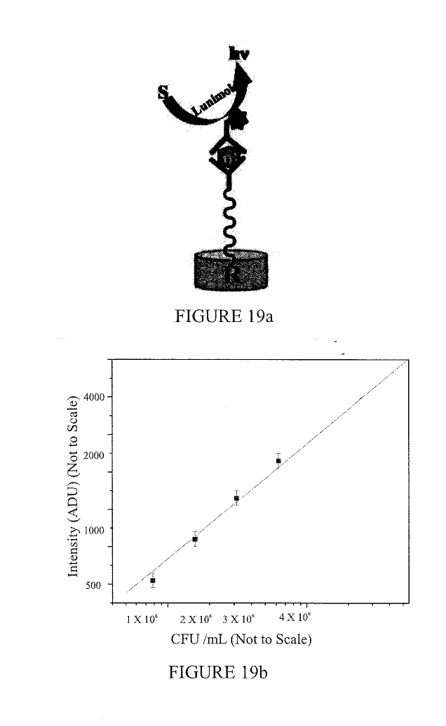

The detection of bacteria is carried out by constructing R—X—R′ self assembled monolayers (SAMs) on the surface of a metal layer in two separate pits. The R′ group was activated by the activation procedure of the EDC/NHS as described in the procedure. A receptor (primary antibody, Ab) specific to E. coli was coupled to the pits via the SAMs. Subsequently, a blank reagent was added to a first pit along with an enzyme labeled secondary antibody (Ab) receptor whereas an analyte (E. coli) containing solution was added to the second pit along with the secondary Ab. After an incubation period, the pits were washed. Subsequent to the washing step, a reagent mixture of luminol and hydrogen peroxide was added and the photon count was accomplished using a detector. The difference between the two pit signals indicates detection and subsequent quantification of the analyte of interest. FIG. 19A illustrates a schematic representation for the detection of E. coli and FIG. 19B illustrates a calibration curve for detection of E. coli in a fluid sample.

In FIG. 19A, R=—SH, —NH₂, —PO₄, —SiO-etc wherein,

S=reagent
Y=primary antibody

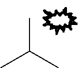

=enzyme labeled secondary antibody
hv=signal

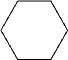

=bacteria, Aflatoxin, spores, whole cell

### Exemplary Experiment 4

### Choline Calibration in Milk

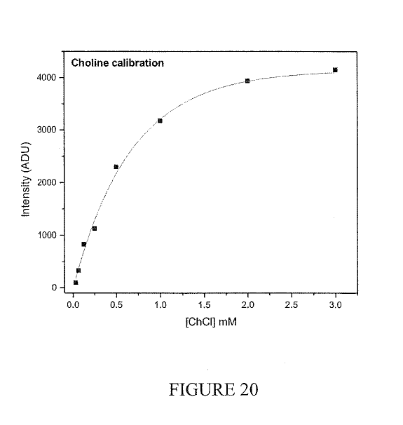

The choline calibration was constructed by spiking a known concentration of choline chloride in diluted milk (0.5% fat containing milk) ranging from 0.0325 to 3 mM (3, 2, 1, 0.5, 0.25, 0.125, 0.0625, 0.0325 mM) in sensor chips containing co-immobilized ChOx and HRP adapted to generate photons. The obtained photon count from the reaction was used for the calibration curve as illustrated in FIG. 20.

### TECHNICAL ADVANCEMENTS AND ECONOMIC SIGNIFICANCE

The technical advancements offered by the process and device of the present disclosure include the realization of:
an ultra-sensitive device for detection of analytes present in small quantity of fluid samples;
a process and device capable of screening and rapidly quantifying analytes present in fluid samples;
an economical process and device capable of rapidly screening and quantifying analytes present in fluid samples with maximum accuracy;
a process and device capable of screening and quantifying analytes present in fluid samples to meet regulatory standards;
a portable device for detection of analytes present in fluid samples;
a simple process and device for detection of analytes present in fluid samples that does not require highly skilled technicians;
a re-usable device for detection of analytes present in fluid samples;
sensor chips and methods for the preparation thereof;
device for evaluating the activity of enzymes and a process for the preparation thereof; and
a method for surface modification of sensor chips by desired chemical agents and/or biological active species.

Specifically, the present disclosure provides:
sensor chips fabricated using Micro-Electro-Mechanical Systems (MEMS) technologies that can screen and accurately determine analytes in fluid samples;
devices that can screen and determine toxic substances in both milk and milk products within a short time period of approx. 15 minutes; the sensor chip was tested for four potent pesticides, MPOx, MP, EPOx and CF and combinations thereof; three different fat content milk samples comprising 0.5% fat, 1.8% fat, and 3.5% fat, were successfully evaluated for pesticide residues with very short analysis time of 15 minutes;
devices that can screen and determine toxic substances in both milk and milk products at very low levels of concentration including concentration level as low as 5 parts per trillion (ppt) in milk;
devices that is portable and can be used in dairy industries and milk collection centers to screen and detect toxic substances in both milk and milk products;
a fabrication process upgradeable on large sized wafers without changing the dimensions on the masks;
eliminating the need for double sided silicon wafers for the fabrication of the sensor chips, resulting in cost saving;
eliminating the need for front-to-back mask aligning for the fabrication of the sensor chips, resulting in cost saving;
very low power consumption by the microheater of the sensor chip, typically about 50 mW to 200 mW for achieving 30° C. to 120° C. temperature range;
successfully cross validating the method for detecting pesticide residues in milk and milk products with the LC-MS/MS technique; and
accomplishing high throughput analysis of pesticide residues in milk and milk products by using heating and non-heating sensor chips.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements, as the use may be in one of the embodiments to achieve one or more of the desired objects or results.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A device for sensing an analyte in a fluid sample, said device comprising:
(i) a sensor chip comprising:
a pre-determined array of pits of varying depth ranging between 0.2 and 30 μm and diameter ranging between 1 mm and 2 mm defined on a silicon-based base comprising a silicon wafer substrate of thickness ranging between 250 and 300 μm, said substrate having at least one oxidized operative surface, and said pits adapted to penetrate said oxidized operative surface and substrate, inner operative surfaces of said pits being oxidized, said metal layer being provided on at least one oxidized operative surface of said pits,
a metal layer of thickness 200-300 nm provided on at least one operative surface of said pits, the metal for said metal layer being at least one metal selected from the group of metals consisting of gold, silver, titanium, rhodium, palladium, platinum and aluminium; and
(ii) a biosensor comprising a cross-linking element fixed to said metal layer, and a set of receptors immobilized on said cross-linking element.

2. (canceled)

3. The device as claimed in claim 1, wherein said base comprises:
a substrate selected from the group consisting of a silicon wafer of thickness ranging between 250 and 300 μm, where at least the operative surface is oxidized and glass;
a nickel layer having a thickness ranging between 300 and 400 nm provided on said substrate to at least partially cover the operative surface of said substrate, said nickel layer being in the form of discrete heating elements with terminals to which leads can be attached for externally powering said heating elements;
an electrical insulating layer deposited on said nickel layer; and
a pit defining layer having thickness ranging between 0.2 and 30 μm provided on said electrical insulating layer;
said metal layer being provided on at least one operative surface of said pits.

4. The device as claimed in claim 1, wherein a chromium layer having thickness ranging between 50-100 nm is disposed between said metal layer and the operative surfaces of said pits.

5. (canceled)

6. The device as claimed in claim 1, wherein said cross-linking element is a compound of formula R—X—R′ where R is selected from the group consisting of thiols (—SH), primary amines (NH₂), silica (SiO₂) and phosphate (PO₄³), X is at least one of a repeating unit having 3 to 18 carbon atoms and R′ is selected from the group consisting of cyanides, thiols, amines and carboxyl.

7. The device as claimed in claim 1, wherein said cross-linking element is at least one selected from the group consisting of L-Cysteine Hydrochloride, Cysteamine Hydrochloride, 3,3′-Dithiodipropionic acid, 3-Mercaptopropionic acid, 6-Mercaptohexanoic acid, 11-Mercapto-1-undecanol, 12-Mercaptododecanoic acid, 15-Mercaptopentadecanic acid, 16-Mercaptohexadecanoic acid, 3-Aminopropyltriethoxysilane, 16-Phosphonohexadecanoic acid, 11-Phosphonoundecanoic acid and 11-Mercaptoundecanoic acid.

8. The device as claimed in claim 1, wherein said receptors are at least one of enzymes, antibodies, antigens, Molecularly Imprinted Polymers (MIPs), aptamers, cells, spores and genetic material.

9. (canceled)

10. The device as claimed in claim 1, wherein said receptors are selected from the group consisting of stabilized choline oxidase, horseradish peroxidase, stabilized Acetylcholinesterase and stabilized Butyrylcholinesterase.

11. A process for making a device for sensing an analyte in a fluid sample, said process comprising the steps of:
providing a silicon-based base having a pre-determined thickness;
forming a pre-determined array of pits on said base; and
depositing a metal layer of thickness 200-300 nm on at least one operative surface of said pits, by at least one of a Direct Current (DC) sputtering and Radio Frequency (RF) sputtering, the metal for said metal layer being at least one metal selected from the group of metals consisting of gold, silver, titanium, rhodium, palladium, platinum and aluminium;
functionalizing said metal layer by using a cross-linking element;
incubating said pits carrying said cross-linking element for a pre-determined period to enable molecular self-assembly of said cross-linking element on said metal layer;
washing said pits with the self-assembled cross-linking element on said metal layer;
activation of said cross-linking element to receive a set of receptors by using a mixture comprising equimolar proportions of 1-ethyl-3-(3-dimethylaminopropyl) carbodiimide and N-hydroxysuccinimide; and
immobilizing said receptors on the activated cross-linking element.

12. The process as claimed in claim 11, wherein the step of forming a pre-determined array of pits further comprises the steps of:
oxidizing at least the operative surface layer of said base comprising a silicon wafer substrate of thickness ranging between 250 and 300 μm;
etching said oxidized operative surface layer and said substrate; and
oxidizing the inner operative surfaces of said pits;
depositing said metal layer on at least one oxidized operative surface of said pits.

13. The process as claimed in claim 11, wherein the step of forming a pre-determined array of pits further comprises the steps of:
depositing a nickel layer having a thickness ranging between 300 and 400 nm on the substrate by at least one of Direct Current (DC) and Radio Frequency (RF) sputtering, to at least partially cover the operative surface of said base comprising a substrate selected from the group consisting of a silicon wafer of thickness ranging between 250 and 300 μm, where at least the operative surface is oxidized and glass;
etching by photolithography, said nickel layer in a targeted manner using a mask to form heating elements having terminals;
providing an electrical insulating layer over said formed heating elements;
providing leads for externally powering said heating elements;
forming a pit defining layer having thickness ranging between 0.2 and 30 μm on said silicon dioxide layer, by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating; and
etching said pit defining layer to form a pre-determined array of pits; and
depositing said metal layer on at least one operative surface of said pits.

14. The process as claimed in claim 11, wherein the step of forming a pre-determined array of pits comprises the step of etching selected from the group consisting of chemical etching, anisotropic etching and photolithographic etching.

15. The process as claimed in claim 11, wherein the step of depositing a metal layer further comprises the step of providing a chromium layer of thickness ranging between 50-100 nm between said metal layer and the operative surfaces of said pits.

16. (canceled)

17. (canceled)

18. (canceled)

19. (canceled)

20. (canceled)

21. A process for evaluating enzyme activity comprising the steps of:
(i) making a device according to claim 1 comprising the steps of:
providing a silicon-based base having a pre-determined thickness, said base being at least one of a silicon wafer substrate of thickness ranging between 250 and 300 μm, where at least the operative surface is oxidized and glass substrate;
depositing a nickel layer having a thickness ranging between 300 and 400 nm on the oxidized operative surface of said silicon-based layer by at least one of Direct Current (DC) and Radio Frequency (RF) sputtering, to at least partially cover said surface;
etching by photolithography, said nickel layer in a targeted manner using a mask to form heating elements having terminals;
providing an electrical insulating layer over said formed heating elements;
providing leads for externally powering said heating elements; and
forming a pit defining layer having thickness ranging between 0.2 and 30 μm on said silicon dioxide layer, by at least one of Direct Current (DC) sputtering, Radio Frequency (RF) sputtering, Plasma Enhanced Chemical Vapor Deposition (PECVD), Low Pressure Chemical Vapor Deposition (LPCVD) and spin coating;
etching said pit defining layer to form a pre-determined array of pits; and
depositing a metal layer of thickness 200-300 nm on at least one operative surface of said pits, the metal for said layer being at least one metal selected from the group of metals consisting of gold, silver, titanium, rhodium, palladium, platinum and aluminium;
functionalizing said metal layer by using said cross-linking element;
incubating said pits carrying said cross-linking element for a pre-determined period to enable molecular self-assembly of said cross-linking element on said metal layer;
washing said pits with the self-assembled cross-linking element on said metal layer;
activation of said cross-linking element to receive the enzyme; and
immobilizing the enzyme under consideration on the activated cross-linking element,
(ii) adding a first reagent adapted to have a specific reaction with the enzyme, said first reagent being adapted to emit photons by a chemical reaction;
(iii) further incubating said pits repeatedly over pre-determined periods of time and conditions so that said first reagent binds with the enzyme to produce a reaction mixture; and
(iv) comparatively studying the photon count emitted from said reaction mixture in said pits with the photon count emitted over said pre-determined periods of time and conditions.

22. A method for detecting at least one analyte in fluid samples, said method comprising the steps of:
(i) a) forming a pre-determined array of first pits on a silicon-based base having a pre-determined thickness, said base constituting a pit defining layer of thickness ranging between 0.2 μm to 30 μm provided on an electrical insulating layer deposited on a nickel layer of thickness ranging between 300 nm and 400 nm adapted to at least partially cover the oxidized operative surface of a silicon wafer substrate of thickness ranging between 250 and 300 μm or the operative surface of a glass substrate, said first pits being coated with a metal layer of thickness 200-300 nm provided on top of an optional chromium layer, the metal for said metal layer being at least one metal selected from the group of metals consisting of gold, silver, titanium, rhodium, palladium, platinum and aluminium, and
providing a first biosensor comprising a first cross-linking element fixed to said metal layer, and a set of first receptors immobilized on said first cross-linking element;
b) forming a pre-determined array of second pits on a silicon-based base having a pre-determined thickness, said base constituting a silicon wafer of thickness ranging between 250 and 300 μm, where at least the operative surface layer is oxidized, said second pits being coated with a metal layer of thickness 200-300 nm provided on top of an optional chromium layer, the metal for said metal layer being at least one metal selected from the group of metals consisting of gold, silver, titanium, rhodium, palladium, platinum and aluminium, and
providing a second biosensor comprising a second cross-linking element fixed to said metal layer, and a set of second receptors immobilized on said cross-linking element;
(ii) adding a fluid sample to at least some of said first pits and incubating for a pre-determined time so that the analyte present in the fluid sample binds with at least some of said first receptors;
(iii) adding a first reagent adapted to have a specific reaction with said first receptors, said first reagent being adapted to emit photons by a chemical reaction;
(iv) further incubating said first pits so that said first reagent binds with at least some of said first receptors which remain unbound during method step (ii) to produce a reaction mixture;
(v) transferring at least a portion of said reaction mixture from said first pits to said second pits;
(vi) adding a second reagent to said second pits to emit photons from said reaction mixture; and
(vii) comparing the photon count emitted from said reaction mixture in said second pits with the photon count emitted by a reference sample.

23. The method for detecting at least one analyte as claimed in claim 22, wherein said first pits and said second pits are either configured on a single sensor chip or on discrete sensor chips, and said pits have varying depth ranging between 0.2 and 30 μm and diameters in the range between 1 mm and 2 mm.

24. (canceled)

25. (canceled)

26. The method for detecting at least one analyte as claimed in claim 22, wherein said nickel layer defines discrete heating elements etched on said oxidized operative surface.

27. (canceled)

28. (canceled)

29. (canceled)

30. (canceled)

31. (canceled)

32. (canceled)
